(19) United States
(12) Patent Application Publication
(10) Pub. No.: US 2013/0131412 A1
(43) Pub. Date: May 23, 2013

(54) RESID CATALYTIC CRACKER AND CATALYST FOR INCREASED PROPYLENE YIELD

(71) Applicants: Robert Mehlberg, Des Plaines, IL (US); Erick D. Gamas-Castellanos, Des Plaines, IL (US); Chad R. Huovie, Des Plaines, IL (US)

(72) Inventors: Robert Mehlberg, Des Plaines, IL (US); Erick D. Gamas-Castellanos, Des Plaines, IL (US); Chad R. Huovie, Des Plaines, IL (US)

(73) Assignee: UOP LLC, Des Plaines, IL (US)

(21) Appl. No.: 13/300,091

(22) Filed: Nov. 18, 2011

(51) Int. Cl. C07C 4/06; B01J 35/02; B01J 29/04

(52) U.S. Cl. 585/312; 585/653; 502/71; 502/73; 502/77; 502/74; 502/69; 502/66; 502/65

(57) ABSTRACT

A process and catalyst for improving the yield of propylene from residual oil feedstock includes obtaining residual oil feedstock from a vacuum distillation tower. The residual oil feedstock has contaminant metals such as sodium or vanadium. The residual oil feedstock is contacted with a cracking catalyst in a catalytic cracking zone to make products. A ZSM-5 zeolite, a binder, a filler and a metal trap are components of the cracking catalyst. The metal trap has a trapping agent in an outer shell of the catalyst, a trapping agent in the ZSM-5 binder or combinations thereof. After reacting, the cracking catalyst is separated from the products in a separator zone, then regenerated by combusting coke deposited on a surface of the cracking catalyst in an oxygen-containing environment. The cracking catalyst is returned to the catalytic cracking zone. The catalyst with the metal trap is also disclosed.

### FIELD OF THE INVENTION

This invention relates to increasing yields of propylene in refinery processes. More specifically, it relates to a process and a catalyst for increasing the yield of propylene from feedstocks high in metal content.

### BACKGROUND OF THE INVENTION

In typical refinery operations, residual oil (“resid”) is produced in a vacuum distillation column. As demand for petroleum fuels and petrochemicals increases, it becomes more important to process feedstocks, such as resids, that are typically low in value. In one process for converting resids to more valuable products, the resid is fed to a resid catalytic cracking zone (“RFCC”) to produce naphtha and distillate fuels which are higher in value. Cracking of the high boiling resid to lighter hydrocarbon streams, such as naphtha or diesel oils, thus generates additional profit due to the relative economic value of the hydrocarbon streams.

One reason resids are difficult to process is because of its high contaminant metals content. Contaminant metals, such as nickel, vanadium and sodium, are known to deactivate the processing catalysts. The metal deactivates the catalyst in one of many ways. In some processes, the contaminant metal is attracted to active reaction sites on the catalyst and physically blocks them. In other cases, the contaminant metal blocks access to pores or cavities in the catalyst support in which the reactions occur. Contaminant metals also reduce the hydrothermal stability of some refining catalysts. Upon exposure to the high temperatures, such as those observed in commercial FCC regenerators, contaminant metals can cause an increased tendency for catalysts to sinter and become amorphous in nature, resulting in a loss of catalyst activity. This is a particular problem in catalytic cracking processes where regenerator temperatures reach as high as 705° C. (1300° F.).

Where a number of catalytic reactions are competing for different sites on the catalyst surface, contaminant metals can alter the catalyst selectivity as well as the activity. Where, for example, a catalyst has both acidic and metallic reaction sites, the contaminant metal could be more attracted to one of these sites over the other. The product distribution will change where the relative activity of one of the two sites changes compared to the other. Thus, both activity and selectivity of the catalyst is altered in the presence of contaminant metals.

Another problem with feedstocks having contaminant metals is that the metals are not removed during catalyst regeneration. Regeneration removes carbon, or “coke”, deposits by combustion. However, this technique does little to remove some contaminant metals.

Methods are known to mitigate the effects of contaminant metals. One basic method is removal of at least some of the contaminant metals from the catalyst. Other methods, known as metals passivation, leave the metals in place, but reduce the negative effects that they have on the activity and selectivity of the catalyst.

### SUMMARY OF THE INVENTION

These and other needs are at least partially addressed by the invention described herein. A process for improving the yield of propylene from residual oil feedstock includes obtaining residual oil feedstock from a vacuum distillation tower. The residual oil feedstock has contaminant metals such as sodium or vanadium. The residual oil feedstock is contacted with a cracking catalyst in a catalytic cracking zone to make products. A ZSM-5 zeolite, a binder, a filler and a metal trap are components of the cracking catalyst. The metal trap has a trapping agent in an outer shell of the catalyst, a trapping agent in the ZSM-5 binder or combinations thereof. After reacting, the cracking catalyst is separated from the products in a separator zone, then regenerated by combusting coke deposited on a surface of the cracking catalyst in an oxygen-containing environment. The cracking catalyst is returned to the catalytic cracking zone.

In another embodiment, the residual oil feedstock is contacted with a first portion of the cracking catalyst in a catalytic cracking zone to make a first slate of products, the cracking catalyst including a ZSM-5 zeolite, a binder, a filler and a metal trap, wherein the metal trap comprises a trapping agent in an outer shell on the surface of the catalyst, a trapping agent in the binder or combinations thereof. The cracking catalyst is separated from the first slate of products, including a light naphtha, in a first separator zone. After separation, the light naphtha is contacted with a second portion of the cracking catalyst to make a second slate of products in an olefin-producing zone. Separation of the second portion of the cracking catalyst takes place in a second separator zone. After separation, the cracking catalyst is regenerated by combusting coke deposited on a surface of the first portion of the cracking catalyst in an oxygen-containing environment and returning the first portion of the cracking catalyst to the catalytic cracking zone and the second portion of the cracking catalyst.

Reducing the effect of contaminant metals has a beneficial effect on yields of light olefins, such as ethylene and propylene. Utilizing a ZSM-5 cracking catalyst with a metal trap prevents the contaminant metals from reducing the activity and altering the selectivity of the cracking catalyst.

### BRIEF DESCRIPTION OF THE DRAWINGS

FIG. 1 is a schematic front plan view of one embodiment of the present resid catalytic cracking process;

FIG. 2 is a schematic front plan view of an alternative embodiment of the present resid catalytic cracking process.

### DETAILED DESCRIPTION OF THE INVENTION

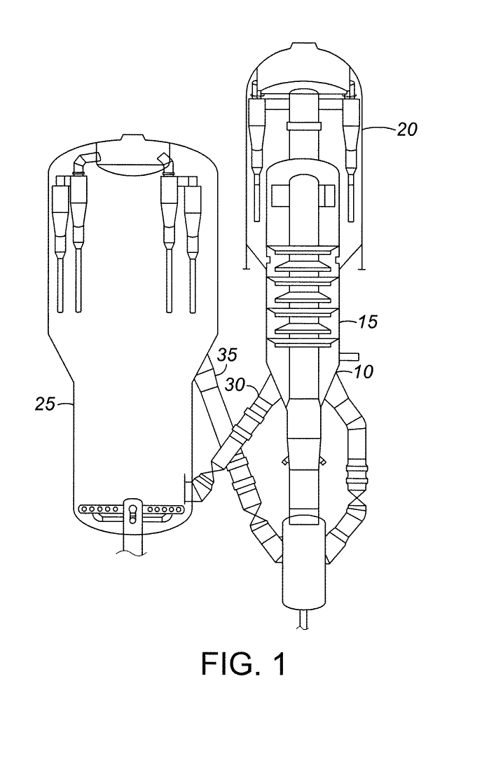

A light naphtha feedstock is obtained from a resid catalytic cracking zone in a process for increasing propylene yield. Any catalytic cracking process or apparatus may be used as the primary cracking zone, including of those adapted to process catalytic cracking feedstocks obtained from atmospheric or vacuum residual oils. An example of this process includes a resid catalytic cracking process (“RFCC”), licensed by UOP, LLC, as shown in FIG. 1.

In some exemplary embodiments, the catalytic cracking zone uses a fluidized bed process. The cracking catalyst is combined with a lift gas and a catalytic cracker feedstock at the entrance to a first riser reactor 10. The catalytic cracker feedstock is a resid having vanadium, sodium or combinations thereof. It is also contemplated that the feedstock contains additional metals, including, but not limited to nickel and zinc. Vanadium is present in amounts of from about 20 ppm to about 400 ppm, preferably in amounts of about 20 ppm to about 150 ppm and more preferably at least 30 ppm. Sodium is present in amounts of from about 1 ppm to about 15 ppm, preferably in amounts of about 1 ppm to about 10 ppm, and more preferably in amounts of at least 5 ppm. Other characteristics of the resid feedstock are: boiling range 340° C. (644° F.) to 566° C. (1050° F.), API in the range of 6 to 21, total sulfur content in the range of 0.3 wt % to 8.0 wt % and microcarbon residue (conradson carbon) in the range of 4.0 wt % to 16 wt %.

As it travels the length of the riser reactor 10, the catalytic cracker feedstock reacts in the presence of the cracking catalyst to generate a first slate of products lower in molecular weight than the catalytic cracking feedstock. Typical products include light cycle oils, naphtha, and a light ends stream. For the purposes of this invention, light naphtha is considered to be C₅-C₆ hydrocarbons from the naphtha, light cycle oil is C₇-C₁₂ hydrocarbons, cycle oils include hydrocarbons heavier than C₁₂ and the light ends stream includes C₄-hydrocarbons.

In the catalytic cracking zone 15, the catalytic cracker feedstock is contacted with a cracking catalyst that promotes cracking of heavy oils to lighter, more valuable, products. Catalysts that promote cracking include, but are not limited to, large and medium pore molecular sieves. The cracking catalyst includes a ZSM-5 zeolite component. U.S. Pat. No. 3,702,886, herein incorporated by reference, describes the ZSM-5 zeolite and its preparation in greater detail. Preferably, the ZSM-5 zeolite is dispersed on a matrix that includes a binder material, such as silica or alumina, and an inert filler material, such as kaolin. These catalyst compositions have a crystalline ZSM-5 zeolite content of 10 to 25 wt-% or more and a matrix material content of 75 to 90 wt-%. Catalysts containing about 25 wt-% crystalline ZSM-5 zeolite materials are preferred. Greater crystalline zeolite content may be used in this catalyst, provided they have satisfactory attrition resistance. The cracking zone catalyst may also comprise another active material such as Beta zeolite.

In addition to the ZSM-5 zeolite, binder and filler, the cracking catalyst also includes a component that acts as a metal trap for the contaminant metals. As described herein, a “metal trap” is a component of the cracking catalyst that stops the contaminant metal from interfering with the catalyst's activity or selectivity. The metal trap acts as a decoy to bind the contaminant metal to the metal trap preferentially over the ZSM-5 zeolite. This limits reduction in activity and selectivity of the ZSM-5 zeolite due to the contaminant metals. The metal trap includes one or more trapping agents as part of the metal trap component. Each of three embodiments of the metal trap is described below. It is contemplated that they be used individually or in any combination with one another.

An embodiment of the metal trap includes the addition of a first trapping agent to the binder of the ZSM-5 catalyst. When present, the first trapping agent is present in amounts of from about 0.5 wt % to about 25 wt %. Possible first trapping agents include cerium, cerium compounds and active alumina. The cerium is present in the form of ceria, CeO₂. Amounts of cerium range from about 0.5 wt % to about 50 wt %, and preferably from about 0.5 wt % to about 15 wt %. A preferred binder for cerium addition is alumina. The first trapping agent is embedded in the binder material by the process of wash coat slurry impregnation.

In another embodiment, the metal trap includes a second trapping agent embedded in a shell on the surface of the cracking catalyst. The second trapping agent includes phosphorous pentoxide, calcium carbonate, ferric oxide or combinations of thereof. When present, the second trapping agent is present in amounts of from about 0.5 wt % to about 30 wt %. Preferably, the calcium carbonate is present in amounts of about 0.5 wt % to about 8.0 wt %, and preferably from about 0.5 wt % to about 3.0 wt %. Amounts of ferric oxide range from about 0.1 wt % to about 30.0 wt % and preferably from about 0.1 wt % to about 10 wt %.

The shell is made of alumina and is prepared by slurry wash coat impregnation and spray drying.

In yet another embodiment of this invention, a third trapping agent is added to the bed of the ZSM-5 catalyst. A commercially available trapping agent is Cat-Aid® V, made by InterCat of Sea Girt, N.J.

Alumina can act as a metal trap, a binder, and/or a support for other components when applied as a shell. If alumina is used as a binder, a layer of alumina can be added on top as a first trap. Preferably, if alumina is used as a binder, ceria would be used as the first trap to take advantage of the higher selectivity of ceria.

Optionally, the cracking catalyst includes a second molecular sieve with a large pore size. The second molecular sieve has pores with openings of greater than 0.7 nm in effective diameter defined by greater than 10- and, typically, 12-membered rings. Pore Size Indices of large pores are above about 31. Suitable large pore zeolite components include zeolites such as X-type and Y-type zeolites, mordenite and faujasite. The second molecular sieve optionally includes a metal ion on its surface, such as a rare-earth metal. Preferably, the rare earth metal is present in amounts of from about 0.1 wt % to about 3.5 wt %. It has been found that Y zeolites with low rare earth content are preferred as the cracking catalyst component. Low rare earth content denotes less than or equal to about 1.0 wt % rare earth oxide on the zeolite portion of the catalyst. Octacat™ catalyst made by W. R. Grace & Co. is an example of a suitable low rare earth Y-zeolite catalyst.

The cracking zone is operated at any useful process conditions. Temperatures range from 510° C. (950° F.) to about 594° C. (1100° F.). Pressures vary between 69 KPa (10 psi) and 276 KPa (40 psi). The space velocity (weight bases) is from about 1 hr⁻¹ to about 50 hr⁻¹. Variations in these conditions are due to differences in feedstock, catalyst and process equipment. Residence time for the catalytic cracker feedstock in contact with the cracking catalyst in the riser is from about 0.1 to 5 seconds, preferably less than or equal to 2 seconds. The exact residence time depends upon the catalytic cracker feedstock quality, the specific catalyst and the desired product distribution. Short residence time assures that the desired products are not converted to undesirable products by further reaction. Hence, the diameter and height of the riser may be varied to obtain the desired residence time.

At the top of the catalytic cracking zone 15, the cracking catalyst is separated from the catalytic cracker feedstock and the lift gas in a first separator zone 20. In a fluidized bed system, some of the cracking catalyst falls by gravity in an area of reduced pressure. One or more cyclones are optionally used to improve separation of the cracking catalyst from the catalytic cracking products.

As the primary catalytic cracking feedstock reacts with the cracking catalyst, coke deposits on the cracking catalyst covering reaction sites and causing a reduction in catalyst activity. The catalyst activity is restored by burning the cracking catalyst in the presence of oxygen from an oxygen source in a primary regeneration zone 25. The first separator zone 20 is in fluid communication with the primary regeneration zone 25, such as using a first conduit 30 to carry the cracking catalyst from the first separation zone 20 to the primary regenerator zone 25. Air is typically used as the oxygen source. As the coke burns, heat and hot combustion gases are generated. Heat generation is regulated by controlling the amount of oxygen, fuel or both provided to the primary regeneration zone. When a substantial portion of the coke has been burned from the cracking catalyst surface, the cracking catalyst is separated from the combustion gases and exits the primary regeneration zone 25. The combustion gases are removed as flue gas. In some embodiments where large amounts of coke are deposited, the cracking catalyst is cooled in a catalyst cooler as it exits the regeneration zone 25. The regeneration zone 25 is in fluid communication with the cracking zone 15, such as via a second conduit 35 that returns the regenerated cracking catalyst to the cracking zone 15.

The product effluent from the primary cracking zone is typically processed through a product recovery section, not shown. Methane, ethane, ethylene, propane, propylene, light naphtha, cycle oil naphtha, cycle oil and gas oil are all potentially part of the first slate of products recovered from the primary cracking zone. The exact products derived from the catalytic cracking process depend on the catalytic cracking feedstock selected, the exact process conditions, the cracking catalyst selected, the downstream processes that are available and the current, relative economic value of the products.

The light naphtha from the product recovery section is provided as feedstock to an olefin-producing zone. In a broad embodiment of the process of this invention, the light naphtha from the catalytic cracker is converted to light olefins in any downstream process. For the purposes of this document, light olefins are defined as C₃-olefins, including ethylene and propylene. By mitigating the effects of the contaminant metals, changes in the activity and selectivity that normally occur when contaminant metals are present in the catalytic cracking zone are reduced. The amount and composition of light naphtha are more favorable toward light olefins production when the effects of contaminant metals are limited.

In another embodiment of this invention, the olefin-producing zone is also a fluidized bed zone. The olefin catalyst is a small particle catalyst. The light naphtha feedstock is contacted with the olefin catalyst in the olefin-producing zone. In a second riser reactor, the olefin catalyst is entrained in the hydrocarbon gases and a lift gas as they move up the riser. At the end of the second riser, a second slate of products, which is now rich in olefins such as propylene, and the entrained olefin catalyst enter a second separation zone and are separated. The olefin-rich product hydrocarbons are drawn from the top of the second separation zone while the olefin catalyst falls away by gravity. Any equipment that can effect such a separation may be used, including, but not limited to, cyclone separators as described above. Following separation of the olefin catalyst, the olefin-rich product exits the second separation zone. The second separation zone is in fluid communication with a regenerator for the olefin catalyst. One example of this fluid communication is a third conduit for transfer of the olefin catalyst from the second separator zone to the regenerator.

The light naphtha feedstock to the olefin-producing zone reacts in the presence of an olefin catalyst. Any known olefin catalyst can be used. In some embodiments of this invention, the cracking catalyst is also used as the olefin catalyst. In other embodiments, the olefin catalyst has a greater selectivity for olefin production than the cracking catalyst and the cracking catalyst has a higher selectivity for cracking than the olefin catalyst.

Any useful process conditions can be utilized in the olefin-producing zone. Temperatures range from 510° C. (950° F.) to about 594° C. (1100° F.). Pressures vary between 69 KPa (10 psi) and 276 KPa (40 psi). The space velocity (weight bases) is from about 1 hr⁻¹ to about 50 hr⁻¹ Variations in these conditions are due to differences in feedstock, catalyst and process equipment. In preferred embodiments, the olefin-producing zone is approximately 34 KPa (5 psi) lower in pressure than the pressure in the catalytic cracking zone. Residence time for the light naphtha feed in contact with the olefm catalyst in the riser is from about 0.1 to 5 seconds, preferably less than or equal to 2 seconds. The exact residence time depends upon the feedstock quality, the specific olefin catalyst and the desired product distribution. Short residence time assures that the desired products, such as light olefins, do not convert to undesirable products in subsequent reactions. Hence, the diameter and height of the riser may be varied to obtain the desired residence time.

Coke builds up on the olefin catalyst as well as the cracking catalyst, and must be burned off to restore catalyst activity. In another embodiment of the invention, the olefin catalyst is regenerated in a second regeneration zone similar to that of the first regeneration zone. Process conditions are selected from the same ranges as the first regeneration zone. The regenerator for the olefin catalyst is in fluid communication with the olefin-producing zone for transfer of the olefin catalyst back to the olefin-producing zone, as by a fourth conduit.

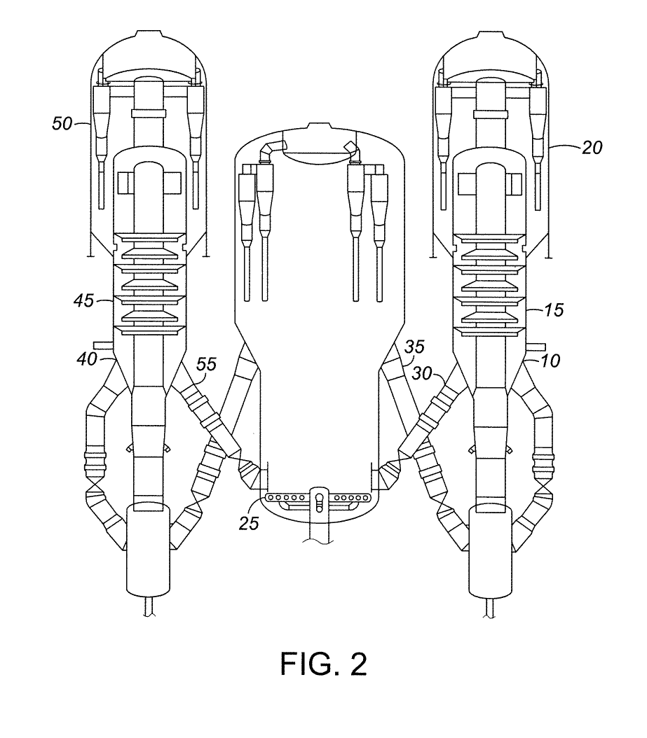

In yet another embodiment of the invention as shown in FIG. 2, the cracking zone 15 and the olefin-producing zone 45 of the second riser reactor 40 use a first portion and a second portion of the same cracking catalyst and share the primary regeneration zone 25. In this case, following regeneration, the first portion of the cracking catalyst is returned to the cracking zone 15. The second portion of catalyst from the primary regeneration zone 25 is fed to the olefin-producing zone 45. In the olefin-producing zone 45, the cracking catalyst contacts the light naphtha feedstock at olefin-producing conditions to make the second slate of light olefin products, such as propylene and ethylene. The second slate of olefin products is separated from the cracking catalyst in the second separator zone 50. The second separator zone 50 is in fluid communication with the primary regeneration zone 25 by a conduit 55 for transfer of the cracking catalyst thereto.

In the primary regeneration zone 25 the first portion of the cracking catalyst comingles with the second portion of the cracking catalyst in a common cracking catalyst pool. Regeneration of the common catalyst pool takes place in the manner described above. Any known catalytic cracking process conditions may be used. Any method may be used to separate the common catalyst pool into a first portion and a second portion. Preferably, the bottom of the primary regeneration zone 25 is shaped so as to divide the common catalyst pool by gravity. It will be understood by those of ordinary skill in the art that “the first portion” and “the second portion” of the cracking catalyst refer to amounts of catalyst only. Once comingled in the common cracking catalyst pool, individual particles are fed to the first portion and second portion of the cracking catalyst randomly.

It is important in this embodiment to mitigate the effects of the contaminant metals. Where there is a common regeneration zone, metals build up on the first portion of cracking catalyst in the cracking zone. However, comingling of the first portion and the second portion in the regeneration zone allows the second portion of cracking catalyst to include catalyst particles that have contaminant metals from prior passes through the cracking zone.

It is to be understood that the features of any of the embodiments discussed above may be recombined with any other of the embodiments or features disclosed herein. While particular features and embodiments of a process and reactor system for increasing propylene yields has been shown and described, other variations of the invention will be obvious to those of ordinary skill in the art. All embodiments considered to be part of this invention are defined by the claims that follow.

What is claimed is:

1. A process for improving the yield of propylene from a light naphtha feedstock, comprising:
obtaining residual oil feedstock from an atmospheric or vacuum distillation tower, the residual oil feedstock comprising contaminant metals;
contacting the residual oil feedstock with a cracking catalyst in a catalytic cracking zone to make a slate of products, the cracking catalyst comprising a ZSM-5 zeolite, a binder, a filler and a metal trap, wherein the metal trap comprises a first trapping agent in the binder, a second trapping agent in an outer shell of the catalyst, a third trapping agent added to the bed of the ZSM-5 catalyst or combinations thereof;
separating the cracking catalyst from the products in a separator zone;
regenerating the cracking catalyst by combusting coke deposited on a surface of the cracking catalyst in an oxygen-containing environment; and
returning the cracking catalyst to the catalytic cracking zone.

2. The process of claim 1 wherein the cracking catalyst comprises an outer shell.

3. The process of claim 2 wherein the shell comprises an alumina wash coat.

4. The process of claim 3 wherein the trapping agent comprises phosphorous pentoxide, calcium carbonate, ferric oxide.

5. The process of claim 1 wherein the trapping agent is present in the binder of the cracking catalyst.

6. The process of claim 5 wherein the trapping agent is cerium or active alumina.

7. The process of claim 1 wherein the trapping agent is added to the bed of the ZSM-5 catalyst.

8. A process for improving the yield of propylene from a light naphtha feedstock, comprising:
obtaining residual oil feedstock from an atmospheric or vacuum distillation tower, the residual oil feedstock comprising contaminant metals;
contacting the residual oil feedstock with a cracking catalyst in a catalytic cracking zone to make a first slate of products, the cracking catalyst comprising a ZSM-5 zeolite, a binder, a filler and a metal trap, wherein the metal trap comprises a first trapping agent in the binder, a trapping agent in an outer shell on the surface of the catalyst, a third trapping agent added to the bed of the ZSM-5 catalyst or combinations thereof;
separating the cracking catalyst from the first slate of products, including a light naphtha, in a first separator zone;
contacting the light naphtha with the cracking catalyst to make a second set of products in an olefin-producing zone;
separating the cracking catalyst from the second slate of products;
regenerating the cracking catalyst by combusting coke deposited on a surface of the cracking catalyst in an oxygen-containing environment; and
returning a first portion of the cracking catalyst to the catalytic cracking zone and a second portion of the cracking catalyst to the olefin-producing zone.

9. The process of claim 8 wherein the metal trap is a trapping agent in the binder and wherein the trapping agent comprises cerium or active alumina.

10. The process of claim 8 wherein the metal trap is a shell and the trapping agent comprises calcium carbonate, ferric oxide, phosphorus pentoxide or combinations thereof.

11. The process of claim 8 wherein the trapping agent is a first trapping agent, further comprising a second trapping agent and wherein the metal trap comprises a combination of the first trapping agent in the ZSM-5 binder and a second trapping agent is in the shell.

12. The process of claim 11 wherein the first trapping agent is active alumina or cerium and the second trapping agent is calcium carbonate, ferric oxide or phosphorus pentoxide.

13. The process of claim 11 wherein the cracking catalyst further comprises a second zeolite component.

14. A catalyst for processing feedstocks high in contaminant metals, the composition comprising:
a ZSM-5 component;
a binder;
a filler; and
a metal trap, wherein the binder comprises a first trapping agent, a second trapping agent in a shell on the outer surface of the catalyst, a third trapping agent added to the bed of the ZSM-5 catalyst or combinations thereof; wherein the first trapping agent is selected from the group consisting of active alumina or cerium and the second trapping agent is selected from the group consisting of calcium carbonate, ferric oxide or phorphorus pentoxide.

15. The catalyst of claim 14 wherein the first trapping agent is cerium.

16. The catalyst of claim 14 wherein the second trapping agent comprises ferric oxide or phorphorus pentoxide.

17. The catalyst of claim 14 wherein the first trapping agent is present in amounts of about 0.5 wt % to about 25 wt %.

18. The catalyst of claim 14 wherein the second trapping agent is present in amounts of about 0.5 wt % to about 30 wt %.

19. The catalyst of claim 14 wherein the shell comprises trapping agents on an alumina wash coat.

20. The catalyst of claim 14 wherein the binder is alumina, silica or silica-alumina.
